(19) United States
(12) Patent Application Publication
(10) Pub. No.: US 2020/0397819 A1
(43) Pub. Date: Dec. 24, 2020

(54) OXYGENATING ORAL AND TOPICAL COMPOSITIONS

(71) Applicant: Daniel A LADIZINSKY, Lake Oswego, OR (US)

(72) Inventor: Daniel A LADIZINSKY, Lake Oswego, OR (US)

(21) Appl. No.: 16/975,269

(22) PCT Filed: Feb. 25, 2019
(86) PCT No.: PCT/US2019/019337

§ 371 Date: Aug. 24, 2020

Related U.S. Application Data
(60) Provisional application No. 62/634,275, filed on Feb. 23, 2018.

(51) Int. Cl. A61K 33/40; A61K 36/48; A61K 36/68; A61K 9/06; A61K 9/14; A61P 1/02; A61P 17/02; A23K 20/20

(52) U.S. Cl. CPC A61K 33/40; A61K 36/48; A61K 36/68; A23K 20/20; A61K 9/14; A61P 1/02; A61P 17/02; A61K 9/06

(57) ABSTRACT

An oral or topical composition includes a peroxide source and a vegetable- or fruit-based peroxide medium configured as a peroxide decomposition catalyst capable of catalyzing decomposition of the peroxide source upon contact with the peroxide source to produce molecular oxygen, the peroxide source and the vegetable- or fruit-based medium being arranged in the composition such that substantial contact of the peroxide source and the peroxide decomposition catalyst is prevented prior to use of the composition.

### CROSS-REFERENCE TO RELATED APPLICATION

This application claims priority to U.S. provisional application Ser. No. 62/634,275, filed Feb. 23, 2018, the disclosure of which is incorporated in its entirety by reference herein.

### TECHNICAL FIELD

The present disclosure pertains to oxygenating oral and topical compositions such as gums, gels, or lozenges. The oral or topical compositions are capable of releasing molecular oxygen in an oral cavity during mastication for promoting the health of the oral cavity or at a site of topical administration for treating a wound or skin disorder or for skin rejuvenation.

### BACKGROUND

The oral cavity is heavily colonized with microorganisms. The gums, cheeks, hard palate, soft palate, gingival crevices between the teeth and the gums, and the teeth each provide areas for bacterial colonization. Sugar, saliva, and presence of the bacteria may lead to tooth decay. Typical remedies for tooth decay include fluoride, fillings, and crowns, in more severe cases a root canal or tooth removal. At present, there is a need for alternative method of inhibiting occurrence, frequency, and severity of tooth decay.

### SUMMARY OF THE INVENTION

The present disclosure pertains to oxygenating oral or topical compositions such as gums, gels, or lozenges. The oral or topical compositions are capable of releasing molecular oxygen in an oral cavity during mastication for promoting the health of the oral cavity or at a site of topical administration for treating a wound or skin disorder or for skin rejuvenation. In various embodiments, the composition is a chewable composition or a composition for topical administration containing peroxide source(s) and catalyst(s) for decomposing the peroxide source(s) to generate molecular oxygen. The generated molecular oxygen is capable of creating an environment with an increased molecular oxygen concentration, which can lessen the ability of S. mutans to use anaerobic glycolysis to produce lactic acid, the primary substance causing degradation of tooth surface enamel. The catalyst and peroxide are kept separate prior to use.

In at least one embodiment, an oral or topical composition is disclosed. The composition includes a peroxide source and at least one vegetable- and/or fruit-based medium configured as a peroxide decomposition catalyst capable of catalyzing decomposition of the peroxide source upon contact with the peroxide source to produce molecular oxygen. The peroxide source and the vegetable- or fruit-based medium are present in the composition in such manner as to prevent substantial contact of the peroxide source and vegetable- or fruit-based medium prior to use of the composition.

The oral or topical composition may be included as a finished product packaged for sale, where the composition may be contained within or covered with a packaging of various embodiments. The packaging may include at least one material configured to substantially prevent light and/or air from reaching the composition, temperature of the composition from increasing, or both.

In an alternative embodiment, a masticatable chewing gum composition is disclosed. The composition is capable of releasing molecular oxygen into an oral cavity during mastication in the mouth. The composition includes: a first component having a gum base, and one of a peroxide source or a vegetable- or fruit-based medium configured as a peroxide decomposition catalyst, the medium catalyzing decomposition of the peroxide source when contacting the peroxide source to produce molecular oxygen; and a second component in contact with the first component and having the other of the peroxide source or the vegetable- or fruit-based medium; wherein the first and second components are arranged to prevent substantial contact of the peroxide and the medium prior to mastication of the chewing gum. The second component may also include a coating that could limit the ability of air or light to reach the peroxide within. The second component may be a coating. The coating may substantially cover an outer surface of the first component. The second component may include a gum base.

The concentration of the peroxide source may be about 0.1 to 0.3% by weight of the composition. The concentration of the medium may be about 0.1 to 3% by weight of the composition. The medium may have particles with an average particle size of less than about 100 μm. The particles may have an example range from about 1 to 1000 μm, 10 to 500 μm, or 50 to 100 μm. Larger and smaller particles are also contemplated. The medium may include a substance derived from lentils, red lentils, bananas, plantains, or a combination thereof.

In yet another embodiment, a method of increasing molecular oxygen concentrations within an oral cavity is disclosed. The method includes masticating a composition of any embodiment disclosed herein over a time period, wherein mastication causes the peroxide source and the vegetable or fruit based medium to come into contact, whereby molecular oxygen is liberated by decomposition of the peroxide source and increases a concentration of molecular oxygen in an oral cavity over a time period. The concentration of molecular oxygen in an oral cavity may temporarily increase to a concentration ranging from about greater than 5 ppm to about 20 ppm of molecular oxygen over a time period.

In an additional embodiment, a method of inhibiting lactic acid generation by microorganisms in an oral cavity is disclosed. The method includes masticating an oral or topical composition of any embodiment disclosed herein over a time period, wherein mastication causes the peroxide source and the vegetable- or fruit-based medium to come into contact with each other such that molecular oxygen is liberated by decomposition of the peroxide source and a concentration of molecular oxygen in an oral cavity is temporarily increased over a time period.

In yet another alternative embodiment, a method for treating a wound or skin disorder or for skin rejuvenation is disclosed. The method includes topically applying an oral or topical composition of any embodiment disclosed herein to a wound or skin and applying moisture to the composition, wound, or skin, wherein the moisture dissolves the composition and causes the peroxide source and the medium to come into contact such that molecular oxygen is liberated by decomposition of the peroxide source and temporarily increases a concentration of molecular oxygen at the wound or skin over a time period. The concentration of molecular oxygen may be capable of diffusing through a surface of the wound or skin from an outside environment.

### DETAILED DESCRIPTION OF THE PREFERRED EMBODIMENTS

As required, detailed embodiments of the present disclosure are disclosed herein; however, it is to be understood that the disclosed embodiments are merely exemplary and may be embodied in various and alternative forms. Therefore, specific structural and functional details disclosed herein are not to be interpreted as limiting, but merely as a representative basis for teaching one skilled in the art.

Throughout this application, where publications are referenced, the disclosures of these publications in their entireties are hereby incorporated by reference into this application to more fully describe the state of the art to which this invention pertains.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps. The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole. The phrase “consisting essentially of” limits the scope of a claim to the specified materials or steps, plus those that do not materially affect the basic and novel characteristic(s) of the claimed subject matter. The terms “comprising,” “consisting of,” and “consisting essentially of” can be alternatively used. When one of these three terms is used, the presently disclosed and claimed subject matter can include the use of either of the other two terms.

Except in the examples, or where otherwise expressly indicated, all numerical quantities in this description indicating amounts of material or conditions of reaction and/or use are to be understood as modified by the words “about.” The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “or” is understood to mean “and/or”.

The terms “percent(s),” “weight percent(s),” “%,”, or “wt. %” are understood to mean percent(s) by weight.

The term “oxygen” is understood to mean molecular oxygen or “02.” Alternatively, the term “oxygen” can also be understood to mean a mixture of different oxygen species including molecular oxygen or molecular oxygen and water.

The oral mucosa makes up a majority of the oral cavity surface colonized with microorganisms, where epithelial cells lining the oral mucosa are continuously shed and replaced. The teeth are another surface within the oral cavity and are notable for development of plaque biofilm since the enamel surface of teeth are the only non-cellular, non-shedding surface for colonization in the oral cavity. The gingival crevice has a lower oxygen concentration than the gingival surface due in part to the continuous flow of gingival crevicular fluid into the crevice. This low oxygen concentration is especially severe in cigarette smokers, who therefore suffer from more severe periodontal disease and premature loss of dentition. The regions between the lips and gums, crevicular spaces, and spaces between the papillae of the tongue all have very low redox potentials and consequently harbor microorganisms that are predominantly of the facultative anaerobic type. In addition, the deep crypts between the papillae provide additional anaerobic niches for colonization. Further, the constant influx of food materials provides nutrients for microbes. Thus, the oral cavity has one of the highest microbial population densities in a mammalian body.

Sugar, saliva, and anaerobic bacteria are a formidable combination that may lead to tooth decay. After eating sugar, particularly sucrose, and even within minutes of brushing the teeth, sticky glycoproteins (combinations of carbohydrates and protein molecules) adhere to the teeth to start the formation of plaque biofilm. At the same time, millions of bacteria known as Streptococcus mutans (S. mutans) also adhere to the glycoprotein. Although many other oral bacteria also adhere, only S. mutans is able to cause cavities. In the next stage, the bacteria metabolize sugars in a glycolysis process. The end product of glycolysis under anaerobic conditions is lactic acid. The lactic acid creates extra acidity (decreased pH) to the extent of dissolving the calcium phosphate in the tooth enamel leading to the start of a cavity.

In addition to tooth decay, anaerobes that are part of the endogenous flora of the oral cavity can be recovered from various infections adjacent to that area, such as infectious cervical lymphadenitis, subcutaneous abscesses, and infected burns in proximity to the oral cavity; infected human and animal bites; paronychia; tonsillar and retropharyngeal abscesses; chronic sinus infection; chronic otitis media; periodontal abscess; infectious thyroiditis; aspiration pneumonia; empyema; and bacteremia associated with one of the above infections. The predominant anaerobes recovered from these infections are species of anaerobic gram-negative bacilli (including pigmented Prevotella and Porphyromonas; Prevotella oralis and other Prevotella species; and Fusobacterium) and gram-positive anaerobic cocci (Peptostreptococcus species), which are all part of the normal flora of the mucosal surfaces of the oral, pharyngeal, and sinus cavities.

There are several antibiotic-based strategies aimed at reducing anaerobic bacterial counts in addition to normal dental hygiene. It is important to note that all antibiotic-based strategies, if used chronically, will lead to resistant flora. Signoretto and Ahn, as disclosed in C. Signoretto et al., “Microbiological evaluation of the effects of hyperbaric oxygen on periodontal disease”, NEW MICROBIOLOGICA 30(4): 431-7 (2007) and S. Ahn, et al., “Effect of oxygen on virulence traits of Streptococcus Mutans”, J. BACTERIOL, December 2007, 189(23), 8519-27, have reduced periodontal disease by providing an external source of molecular oxygen using perfluorocarbons and even hyperbaric oxygen, but a need for a simpler method remains. Others have demonstrated some efficacy of a chewing gum containing urea hydrogen peroxide. H. Etemadzadeh, “Plaque growth inhibiting effect of chewing gum containing urea hydrogen peroxide,” J. CLIN. PERIODONTAL, 18(5), 337-40 (May 1991).

The use of urea peroxide (“carbamic peroxide”) in chewing gum is also disclosed in U.S. Pat. No. 5,500,207 to Goulet along with other peroxides, for teeth whitening. U.S. Pat. No. 5,972,374 to Theisen discloses a cylindrical chewing gum with separable portions, the cylinder having a central area containing a tooth whitening agent which may contain carbamic peroxide. U.S. Pat. No. 5,693,334 to Mishewitz discloses slow release gum formulations containing encapsulated sodium bicarbonate and a peroxygen compound such as carbamic peroxide. U.S. Pat. No. 5,908,614 to Montgomery discloses an oral care composition which contained a hydrogen peroxide precursor and an activator which stimulates production of peroxidate enzyme in the oral cavity to generate hydrogen peroxide from the peroxide precursor.

Except for the methods disclosed by Signoretto and Ahn, which elevate the oxygen tension in the oral cavity, by means clearly not useable in the absence of a clinical setting, the other references use peroxides for whitening or as biocides.

A chewing gum or equivalent device that produces molecular oxygen in the oral cavity, as disclosed herein, will be beneficial to dental and oral cavity health by changing the anaerobic microenvironment of the periodontal region. This will inhibit the anaerobic bacteria and therefore the deleterious effects of their anaerobic metabolic byproducts such as lactic acid. It has been observed that the ability of S. mutans to form biofilms is severely impaired by oxygen, as disclosed by Ahn. Since S. mutans is a facultative anaerobe, it likely will not be eliminated by elevated oxygen levels. But if it switches to aerobic metabolism rather than anaerobic glycolysis, the detrimental lactic acid byproduct will not be present to the same degree, and there will be reduced effects on the dental enamel. Also, reduction in anaerobic bacteria may reduce infectious or inflammatory conditions elsewhere in the body.

Additionally, a constant and adequate oxygen supply is important for cell and tissue homeostasis. It is documented that oxygen can play a role in energy production, cell membrane maintenance, mitochondrial function, and cellular repair. Atmospheric oxygen is taken up by the epidermis. Physical injury to skin can compromise the arterial, venous, or capillary systems of tissue, which in turn may cause hypoxia and ischemia. The tissue repair process requires an increased metabolic activity of a variety of cells, resulting in a high oxygen demand. Recent research has demonstrated that increased oxygen tension in a wound promotes wound healing by stimulating several processes, including phagocytosis (engulfing of microorganisms, cells, or debris by macrophages or neutrophils), degradation of necrotic wound tissue, collagen production, neovascularization, and neutrophil-mediated oxidative microbial killing. Thus, a composition that produces molecular oxygen at a site of a wound or skin injury may be beneficial to healing or rejuvenation.

In one or more embodiments, a composition solving one or more problems recited above is disclosed herein. The composition may be in the form of a masticatable gum, wax, gel, or the like, all termed collectively “chewable composition” hereafter, unless noted otherwise. The compositions of various embodiments can include chewing gums, the formulations of which are well known, as illustrated by U.S. Pat. Nos. 6,696,043; 5,405,623; 5,1992,562; 5,085,872; 5,145,696; and 4,986,991, all of which are incorporated herein by reference. The oral composition of various embodiments may also be in the form of a lozenge which may slowly dissolve in the oral cavity, with or without chewing.

The composition may be gum-based. Traditionally, the chewing gum base may be a natural gum based on rubber latex, or a variety of synthetic polymers such as homo- and co-polymers based on polyvinyl acetate, with comonomers such as ethylene, vinyl propionate, and vinyl laurate. Chewing gum bases are typically natural products, usually extracts from certain trees, plants, and microbes. They are usually divided into three categories: soft, medium, and hard. The soft and medium varieties are preferred for use in the chewing gum formulations herein. The chewing gum bases commonly used in conventional chewing gum are suitable as ingredients of the formulations of the preferred embodiment. In one embodiment, the chewing gum base is added at between 50 and 90%, including 55%, 60%, 65%, 70%, 80%, and 85% by weight. In a further embodiment, the chewing gum base is added at between 65% and 85%. In a further embodiment, the chewing gum base is added at between 70% and 80%. It is to be understood that substantial quantities of chewing gum additives are used, the concentration may vary.

The composition may include one or more sources of a peroxide. The one or more sources of peroxide may form a first portion of the composition. The portion may relate to a layer or a portion of a packaging, separating the first portion from the second portion to prevent substantial contact of the first portion from the second portion. The peroxide source may be any peroxide such as solid or liquid peroxide, which is safe for human and/or animal consumption and which can liberate molecular oxygen in the presence of a suitable catalyst. The composition may include a plurality of different peroxide sources or 1, 2, 3, 4, 5, 6, 7, 8, 9, 10, or more different peroxides sources.

Solid peroxides may include various percarbonates and perborates such as, for example, sodium percarbonate, carbamic peroxide, and calcium peroxide. Liquid peroxides may also be used. An non-limiting example of a liquid peroxide is hydrogen peroxide. When liquid peroxides are used, the liquid peroxide may be present in the composition in an encapsulated form with a water soluble or biodegradable polymer. Examples of water soluble or biodegradable polymers include polyvinyl alcohol, polyvinylpyrollidone, or a natural coating such as crosslinked or non-crosslinked gelatin. Methods of encapsulating liquids with such coatings are known in the art, and are disclosed, for example, in U.S. Pat. Nos. 5,908,614 and 5,693,334, which are both incorporated herein by reference. “Liquid peroxides” as used herein also include solutions of solid peroxides.

As was stated above, the peroxide source may include sodium percarbonate, carbamide peroxide, calcium peroxide, or a combination thereof.

Sodium percarbonate is a relatively stable complex containing 2 moles of sodium carbonate complexed with 3 moles of hydrogen peroxide (27% hydrogen peroxide by weight). It is highly water soluble (120 grams per liter at 20° C.) and produces a pH upon dissolution of between 10 and 11 (for a 1% solution). Thus, although sodium percarbonate possesses the desirable hydrogen peroxide-releasing properties, alone they are of little utility for the activation of a peroxidase enzyme due to their high in-solution pH properties. Accordingly, a pH adjusting may be utilized to normalize the pH to a range of about 4.0-7.9.

Carbamide peroxide, a topical anti-infective oral health agent, is a 1 to 1 molar complex between urea and hydrogen peroxide (35% hydrogen peroxide by weight) with a molecular weight of 94.07. Hydrogen peroxide and urea are classified “Generally Recognized As Safe” (GRAS) by the United States Food and Drug Administration (FDA), with no maximum allowable limit. Urea is used as a formulation or fermentation aid in yeast-raised bakery products, in alcoholic beverages, and in gelatin products. In a non-limiting example, the composition of various embodiments includes about 0.29% by weight of carbamide peroxide. Carbamide peroxide is prescribed to treat canker sores and other minor inflammatory conditions of the gums and mouth. It is a common additive to tooth whitening products. The most serious adverse reaction to carbamide peroxide is local irritation. The health opinion written by the European SCIENTIFIC COMMITTEE ON COSMETIC PRODUCTS AND NON-FOOD PRODUCTS states that the content of hydrogen peroxide in tooth whitening products should not exceed 3.6% (10% carbamide peroxide). Tooth whitening products containing more than 0.1% hydrogen peroxide (0.3% carbamide peroxide) are thus recommended to be administered under supervision of a dentist. Carbamide peroxide is usually manufactured in the form of crystals which are highly soluble in water (800 grams per liter of water at 20° C. to yield a saturated solution of 44.4% carbamide peroxide, equivalent to a hydrogen peroxide concentration of 15.5%). When carbamide peroxide is solubilized in water, a pH of 3.40 (for a saturated solution) to 4.05 (for a 1% solution) is obtained. This pH is slightly below the desirable range, for activating a peroxidase enzyme in the aqueous contact solution absent a pH adjusting agent, so a pH adjusting agent may be added.

The composition may have the pH adjusted to not be too basic or too acidic or to avoid irritation of the lining of the oral cavity and the tongue. Furthermore, when biological enzymes are used as peroxide decomposition catalysts, each enzyme may have a pH range that is most effective. Thus, the chewable composition may contain basic substances such as sodium or potassium carbonates or bicarbonates, calcium carbonate, calcium or magnesium hydroxide, alkali metal phosphates and hydrogen phosphates, alkali metal acetates and propionates, etc., to lower acidity, and acidic substances such as alkali metal dihydrogen phosphates, ammonium halides and sulfates, carboxylic acids such as acetic acid, propionic acid, and mineral acids in minor quantities, to lower basicity. Buffer preparations are also useful in such compositions.

In various embodiments, the peroxide source is at least or is about 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7%, 8%, 9%, 10%, 11%, 12%, 13%, 14%, 15%, 16%, 17%, 18%, 19%, or 20% by weight of the composition. In various embodiments, the weight percent of the at least one peroxide source in the composition may be a range between any two weight percents listed above.

For example, the composition may include 1% to 20% by weight hydrogen peroxide. In other non-limiting examples, the composition may include about 3% to 7% by weight calcium peroxide including about 3.4%, 3.5%, 4%, 4.5%, 4.6%, 5%, 5.5%, 6%, or 6.5% by weight calcium peroxide. In other examples, the peroxide sources can also be mixed and include about 2, 3, 4, or more different peroxides in any ratio. In examples, the composition of various embodiments can include a mixture of calcium peroxide and zinc peroxide, where the zinc peroxide is at a concentration of about 0.3 to 0.7% by weight.

The composition includes a peroxide decomposition catalyst. The decomposition catalyst forms a second portion, which may relate to a layer or another part of a packaging of the composition, ensuring no substantial contact of the first portion with the second portion until such contact is desirable.

The catalyst may be a vegetable- and/or fruit-based medium. While fruit or vegetable is mentioned, other botanical materials such as legumes are expressly included. The catalyst is capable of catalyzing decomposition of the peroxide source when contacting the peroxide source to produce molecular oxygen. Examples of the medium may include red lentils, other lentils, bananas, plantains, or a combination thereof. The catalyst may also be a naturally occurring or synthetic enzyme such as catalase, superoxide dismutase, salivary peroxidase, myeloperoxidase, glutathione peroxidase or other, or an inorganic catalyst such as manganese dioxide, or an alkali or alkaline earth permanganate or other. The catalyst can include catalase. Catalase has low toxicity characteristics and high stability that may be beneficial to the composition. The catalyst may be incorporated in the composition in any form suitable for use by a subject, where examples of such forms can include powders, extracts, solutions, emulsions, crystals, particles, dried/dehydrated forms, etc.

Lentils, also known as Lens culinaris or Lens esculenta, relate to lens-shaped seeds of legume family. Lentils represent a dietary staple of several world regions, especially South Asia. This application contemplates utilization of any lentil variety, especially red lentils.

Red lentils have many health benefits and are an excellent source of natural enzymes including catalase, an enzyme capable of stabilizing free radicals, which can break down hydrogen peroxide into water and molecular oxygen. The red lentils-catalyzed decomposition reactions may result in elevated levels of molecular oxygen generation. Red lentils are also an inexpensive ingredient.

In at least one embodiment, a combination of lentils may be used. For example, the combination may include the following types of lentils: Brewer's, Beluga, Brown/Spanish pardina, French green, Puy lentils, dark/light green, Indianhead, Yellow/tan lentil, Red Chief, Eston Green, Richlea, Laird, Mansoor, Petite crimson/red, Macachiados, or a combination thereof.

As was mentioned above, the lentils may be used as an extract from fresh or dried seeds. Alternatively, the lentils may be used in a form of paste, pulp, flour, or in another form. The lentils component may be prepared from skin only, the “meat” or content found within the skin, or both. The lentils may be used raw or thermally treated such as by cooking, steaming, baking, or frying. The lentils may be used ripe or unripe.

In addition to, or alternatively to, lentils, the medium may include bananas, plantains, or both. The medium may contain any type of banana or plantain from the Musa genus, for example Musa acuminata Colla, Musa balbisiana Colla, and Musa×paradisiaca L. The medium may include Cavendish bananas, Manzano or Apple bananas, Fig bananas, Lady's Finger bananas, Pi sang Raja bananas, Red bananas, Cuban red bananas, Orinoco bananas, Blue Java Ice Cream bananas, False Horn bananas, Praying Hands or Benedetta bananas, Hawaiian plantains,

The medium may contain any part of the banana or plantain plant, for example the leaves, the flower, the fruit and/or its portions such as “meat,” skin or peel, the seeds, or a combination thereof. The medium may include the fruit as pulp, flour, liquid, or a combination thereof. The banana or plantain may be mashed, liquified, thermally processed such as cooked, grilled, steamed, boiled, roasted, fried, or baked, or otherwise prepared to form a portion or entirety of the medium. The banana or plantain may be used unripe when the peel is green or in various stages of ripeness the banana is ripe such that the peel is yellow or overripe such that the peel is brown. For example, the medium may contain only unripe banana, only ripe banana, only overripe banana, or combination thereof.

The medium may include organic lentils, bananas, plantains, or a combination thereof, where the term “organic” is defined, for example, by regulatory agencies of the United States of America such as the FDA or of the European Union.

In various embodiments, the medium may be in the form of particles. The particles may have an average particle size of at least or of about 1 micron, 5, microns, 10 microns, 20 microns, 30 microns, 40 microns, 50 microns, 60 microns, 70 microns, 80 microns, 90 microns, 100 microns, 500 microns, or 1000 microns. In various embodiments, the average particle size is a range between any two particle sizes listed above.

In various embodiments, the catalyst is at least or is about 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7%, 8%, 9%, 10%, 11%, 12%, 13%, 14%, 15%, 16%, 17%, 18%, 19%, or 20% by weight of the composition. In various embodiments, the weight percent of catalyst in the composition is a range between any two weight percents listed above.

Optional but desirable additives to the formulation which may have beneficial effects on the rate of decomposition of the peroxide source by the catalyst during the mastication process include, but are not limited to, hydroxides, oxides, salts of alkaline earth metals, particularly carbonates; hydroxides and carbonates of sodium, calcium and potassium; silicas; and calcium silicate. In non-limiting examples, the composition includes at least about 0.5% or about 0.5% to 6% by weight of calcium carbonate, including at least or including about 1%, 1.5%, 2%, 2.5%, 3%, 3.5%, 4%, 4.5%, or 5% by weight calcium carbonate. The composition may include a weight percent of calcium carbonate that is a range between any two weight percents listed above.

In various embodiments, the composition may include a gum base that is nondigestible and is a part of any chewing gum. The gum base of various embodiments may include a resin, wax, or elastomer. Resins such as terpene may be the chewable portion. Waxes may soften the gum. One or more elastomers may be added to increase flexibility of the gum base. The molecular composition of the gum base may be very similar to that of plastics and rubbers. Gum base is nondigestible and can be a part of any chewing gum known to impart characteristics that are associated with chewing gums.

Alternatively, the chewing gum base may be a natural gum based on chicle or rubber latex, or a variety of synthetic polymers such as homo- and co-polymers based on polyvinyl acetate, with comonomers such as ethylene, vinyl propionate, and vinyl laurate. Chewing gum bases are typically natural products, usually extracts from certain trees, plants, and microbes. They are usually divided into three categories: soft, medium, and hard. The soft and medium varieties are preferred for use in the chewing gum formulations herein. The chewing gum bases commonly used in conventional chewing gums may be also suitable as ingredients of the formulation. The chewing gum base may be added at between about 50 and 90%, including about 55%, 60%, 65%, 70%, 80%, and 85% by weight of the composition. In further embodiments, the chewing gum base may be added at between about 65% and 85%. In a further embodiment, the chewing gum base may be added at between about 70% and 80%. It is to be understood that substantial quantities of chewing gum additives may be used, the concentration of each ingredient may vary.

The composition may further include one or more conventional excipients such as sweeteners, sweetness enhancers, sugar alcohols, flavors or flavorings, coloring agents, vitamins, inorganic fillers, surfactants, oils, emulsifiers, thickening agents, stabilizers, polymers, humectants, biogenic active substance, antioxidants, deodorants, antimicrobial agents, anti-caking agents, nutritional supplements, etc.

Sweeteners or sweetness enhancers may be added to the chewing gum including sweeteners such as sucrose, fructose, glucose, high fructose corn syrup, corn syrup, xylose, arabinose, rhamnose, erythritol, xylitol, mannitol, sorbitol, inositol, acesulfame potassium, aspartame, neotame, sucralose, saccharine, or combinations thereof. An example natural sweetener may be a monkfruit (Siraitia grosvenorii) which is a herbaceous perennial vine of the Cucurbitaceae family, native to southern China and northern Thailand.

The sweetness enhancer may be selected from naringin dihydrochalcone, mogroside V, swingle extract, rubusoside, rubus extract, rebaudioside, raw honey, maple syrup, molasses, coconut sugar, and stevioside. Although natural sweeteners such as sucrose, maltose, glucose, fructose, etc., may be used, these sweeteners may provide a further energy source for S. mutans, which is undesirable. An artificial sweetener may thus be used while not supplying a further energy source for S. mutans.

The sweetener or sweetness enhancer may be added at between about 0.2 and 10%, based on the weight of the composition, including about 0.5%, 0.7%, 1%, 1.5%, 2%, 2.5%, 3%, 4%, 5%, 6%, 7%, 8%, and 9%.

Some of the sweeteners or sweetness enhancers may be used not only for increasing sweetness of the composition for a consumer's enjoyment, but for additional properties of the specific sweeteners or sweetness enhancer. For example, xylitol may assist in reduction of an oral biofilm plaque. Xylitol is a naturally occurring alcohol found in most plant material, including many fruits and vegetables. Xylitol is widely used as a sugar substitute and in “sugar-free” chewing gums, mints, and other candies. Xylitol is added to some chewing gums and other oral care products to prevent tooth decay and dry mouth. Xylitol tastes sweet but, unlike sugar, is not converted in the mouth to acids that cause tooth decay. Xylitol may reduce levels of decay-causing bacteria in saliva and act against some bacteria that cause ear infections. Research has shown that a safe dosage of Xylitol as a pharmaceutical for most adults can range up to about 50 grams per day. Research has also shown that use of xylitol-containing products such as foods, chewing gum, candies, and toothpaste that provide 1-20 grams of xylitol per day may significantly reduce the rate of cavity formation in both adults and children. The composition may include about 1, 2, 3, 4, 5, 6, 7, 8, 9, 10, 15, 20, 30, 40, 50, 100, 150, 200, 300, 350, 400 or more mg of xylitol.

The composition may include artificial synthetic sweeteners such as aspartame. Aspartame is an artificial sweetener made from a combination of two amino acids, aspartic acid and phenylalanine. Aspartame may be included in the amount of or up to about 0.5, 1, 1.5, 2, 2.5, 3, 3.5, 4, 4.5 5, 5.5, 6, 6.5, 7, 7.5, 8, 8.5, 9, 9.5, 10, 15, 20, 25, 30, 35, 40 or more mg, which complies with a recommended dose of aspartame in an average adult of 70 kg of 2800 mg/day.

An alternative artificial sweetener may be acesulfame. The FDA has set its acceptable daily intake level (ADI) for acesulfame at 33 mg/kg per day, or for an average 70 kg adult, 2310 mg/day. The composition may include up to or about 0.25, 0.5, 0.75, 1, 1.25, 1.5, 1.75, 2, 2.25, 2.5, 2.75, 3, or more mg of acesulfame.

In various embodiments, the composition may include sucralose. Sucralose is a synthetic derivative of sucrose. The FDA has set an acceptable daily intake level (ADI) of sucralose at 5 mg/kg per day, or 350 mg in an average 70 kg adult. The composition may include about 0.5, 07, 1, 1.2, 1.5, 1.7, 1.9, 2.0, 2.1, 2.3, 2.5, 3, 4, 5, 6, 7, 8, 9, 10 or more mg sucralose.

Alternatively, or in addition, the composition may include a stevia-based sugar substitute. A stevia-based sugar substitute may be made of rebiana, a purified extract of stevia leaf, erythritol, and natural flavors. FDA has set an ADI of Truvia®, an example stevia-based sugar substitute, at 4 mg/kg per day, or 280 mg for an average 70 kg adult. The composition may include up to or about 0.5, 0.75, 1, 1.5, 2, 2.5, 3, 3.5, 4, 4.5 mg.

The composition may further include sugar alcohols, which refer to any of the acyclic linear polyhydric alcohols derived from carbohydrates. Examples of sugar alcohols include mannitol or sorbitol. Sorbitol or glucitol is a slow-metabolizing sugar alcohol derived from fruits, corn, and seaweed. Polyols, including sorbitol, are resistant to metabolism by oral bacteria which break down sugars and starches to release acids that may lead to cavities or erode tooth enamel. They therefore do not contribute to tooth decay. The usefulness of polyols, including sorbitol, as alternatives to sugars and as part of a comprehensive program including proper dental hygiene has been recognized by the American Dental Association. The FDA has approved the use of a “does not promote tooth decay” health claim in labeling for sugar-free foods that contain sorbitol or other polyols.

Flavors or flavoring and coloring agents may be added to enhance the acceptance or appeal of either or both parts, or as indicators of the reactivity of peroxide and the progress of radical oxygen generation. The most desirable flavors may include, among others, food grade orange, lemon, peppermint, spearmint, mint, bubble gum, cherry, watermelon, strawberry, and apple varieties. As coloring agents FD&C or FD&C water soluble dyes may be used; FD&C Blue #1 and FD&C Blue #2 may be included. The flavor additive may be peppermint oil added at between about 0.05 and 0.5%, including 0.075%, 0.15%, 0.2%, 0.3%, 0.4%, and 0.45%. In a further embodiment, the peppermint oil or other flavor additive may be added at between about 0.1 and 0.4%. Although it is to be understood that the strengths of flavoring may differ considerably, a much lower or higher concentration may be needed. Various examples of flavors include natural and artificial flavors chosen from synthetic flavor oils and flavoring aromatics, and/or oils, oleo resins and extracts derived from plants, leaves, flowers, fruits, seeds, and so forth, and combinations thereof. Non-limiting representative flavoring agents include: almond oil, amaretto flavor, anise oil, natural apple flavor, apricot flavor, banana creme flavor, bavarian creme flavor, vanilla extract, black walnut flavor, blackberry flavor, blueberry flavor, brandy flavor, bubble gum flavor, butter flavor, butter rum flavor, butterscotch flavor, caramel flavor, champagne flavor, cheesecake flavor, cherry flavor, chocolate flavor, chocolate hazelnut flavor, cinnamon oil clove oil, natural coconut flavor, coffee flavor, cotton candy flavor, cran-raspberry flavor, cranberry flavor, creme de menthe flavor, eggnog flavor, English toffee flavor, chili flavor, tart & sour flavor, ginger oil, natural grape flavor, grapefruit oil, pink, natural guava flavor, honey flavor, horehound flavor, coffee flavor, lemon oil, natural lemonade flavor, licorice flavor, lime oil, natural mango flavor, maple flavor, marshmallow flavor, menthol, eucalyptus flavor, mint chocolate chip flavor, nutmeg oil, natural orange cream flavor, orange oil, natural peach flavor, pecan flavor, peppermint oil, pina colada flavor, pineapple flavor, pistachio flavor, plum flavor, praline and cream flavor, praline flavor, pumpkin flavor, raspberry flavor, red licorice flavor, root beer flavor, salt water taffy flavor, sassafras flavor, spearmint oil, natural strawberry flavor, tangerine oil, natural teaberry flavor, tropical punch flavor, tutti-frutti (passion fruit) flavor, vanilla butternut flavor, watermelon flavor, wintergreen oil, etc. In one example, the composition of various embodiments includes spearmint, peppermint, vanilla, and menthol flavors.

Examples of anti-caking agents may include cellulose, microcrystalline cellulose, potato starch, corn starch, rice flour, calcium silicate, calcium stearate, calcium phosphate, calcium sulfate, silicon dioxide, sodium silico-aluminate, etc.

Examples of nutritional supplements may include various vitamins or dietary supplements such as zinc gluconate. Regarding zinc gluconate, zinc is a supplement with multiple biological functions and recommended daily allowance for zinc is 11 mg for adults, with maximum allowable dose of 40 mg per day. Zinc sulfate is the least expensive but also least soluble form and is poorly absorbed compared to zinc gluconate. Zinc gluconate has been shown to have effects on reducing bad breath. Volatile Sulphur compounds (VSCs) produced by anaerobic microorganisms on the tongue are major contributors to oral malodor. Antimicrobial agents such as zinc salts may therefore indirectly reduce the production of VSCs. One study evaluated the effect of four zinc-containing sorbitol lozenge formulations (0.1 0.5% zinc gluconate) on oral malodor (‘morning breath’) by breath and tongue flora analysis on 24 healthy volunteers. Chlorhexidine (0.2% chlorhexidine gluconate) mouthwash was used as a positive control and sorbitol lozenges as a negative control. All treatments were effective in reducing sulfides in breath odor but chlorhexidine and 0.5% zinc lozenges produced the greatest reduction. All treatments produced a significant decrease (p<0.001) in bacterial counts 15 min post treatment, with chlorhexidine being most effective. The composition may include up to or about 0.05, 0.075, 0.1, 0.15, 0.2, 0.25 wt. % zinc gluconate, based on the weight of the composition, which is within a weight percent range effective for treating for halitosis or bad breath.

In the topical form of the composition discloses herein, the gum-based ingredients may be absent, especially the gum base, sweeteners, flavoring agents, colorants, etc. Instead, additional components such as petroleum jelly may be added to provide a spreadable base for the topical composition. Any mixture of hydrocarbons which may be used as a lubricant, gel, or ointment may be included and form a first portion, second portion, or both of the topical formulation described below. Example components for the topical composition may include lard, butter, various oils such as coconut oil, sunflower oil, palm oil, almond oil, olive oil, avocado oil, argan oil, jojoba oil, other oils, but also propolis, and their combinations.

The peroxide and catalyst are separated and are kept separated in any manner to substantially prevent premature oxygen generation that can result in a short shelf life. Any suitable separating method may be used. Thus, any substantial contact of the peroxide and the catalyst prior to use is prevented. By the term “substantial contact” as used herein is meant a degree of contact which provides for a storage stable product. The composition may limit decomposition to at most about 10% of the peroxide source when stored at ambient temperature (20-25° C.) and about 50% relative humidity for one month. The composition may limit decomposition to about 10% of the peroxide source over about a 6-month period or longer. The composition is stable to peroxide decomposition by the catalyst.

Alternatively, the peroxide and the catalyst may be in contact prior to use as long as the peroxide and the catalyst are kept in a dry environment, preventing moisture from initiating the catalysis. The catalysis occurs upon moisture exposure which allows the two soluble molecules to contact one another in an aqueous environment which leads to the catalysis of H₂O₂ into 02 and H₂O. Thus, a contact between the peroxide and the catalyst has to be eliminated or prevented in a moist, wet, or aqueous environment. The “dry environment” relates to relative humidity of 35% or less, 30% or less, 25% or less, 20% or less, 15% or less, 10% or less, or 5% or less. The “aqueous environment” relates to relative humidity of 35% of more.

To limit moisture in a packaging, where the peroxide and the catalyst are kept in contact, a desiccant, a hygroscopic substance used a drying agent, may be included. Any food-safe desiccant may be used. The desiccant may be incorporated as integral or separate part of the packaging. Additionally, during the packaging process, for example into individual blister packages, a nitrogen flush may be used to reduce ambient oxygen.

The peroxide and the catalyst may be encapsulated in a variety of ways. For example, the encapsulating material may form a coating or wall around the component to be encapsulated. The thickness of the wall may be adjusted to account for the processing methods used to prepare the oral compositions. For example, in a highly viscous gum base, the walls of the encapsulant may constitute from about 20 to 40 weight percent by weight of the encapsulated material, by way of example and not by way of limitation, whereas in less viscous gums, in gels, or in lozenges where less shear and less pressure during processing is to be expected, much thinner walls may be appropriate, even approaching a mono- or bi-molecular layer. The encapsulant is selected such that it is not soluble by the oral composition ingredients. In the case of dissolvable tablets or lozenges, the encapsulant should be water soluble. A non-limiting example of an encapsulant may be carnauba wax since it will not put the carbamide peroxide into solution. Suitable methods may be found, for example, in S. J. Risch, ENCAPSULATION AND CONTROLLED RELEASE OF FOOD INGREDIENTS, ACS Symposium Series, Vol. 590 © 1995, American Chemical Society, ISBN 13; 9780841231641, and other references.

The composition may be gum in the form of “sticks,” flat strips, granules, etc. Processes typically used to produce a gum strip or stick include extrusion through a die. Yet, the typical processes require mixing the ingredients at 60° C. with a heated mastic that can denature enzymes and proteins. Such processes would damage and compromise the hereby disclosed ingredients such as the peroxide and the catalyst.

Thus, unlike conventionally prepared gum, the composition disclosed herein is prepared in a relatively low temperature, dry environment. The gum contains volatile active ingredients, the peroxide and the catalyst, which need to be protected from conditions such as elevated temperature of about 60° C. or higher and moisture. Therefore, the composition may be prepared, for example, in a cold press or another compression machine, for example used in the manufacture of pharmaceuticals, to prevent inactivation since heat is not needed. The cold press may be conducted at relatively low or ambient temperature of 20° C. to 25° C. or at temperatures of 15 to 25° C., 18 to 23° C., or 19 to 22° C. The “dry environment” relates to relative humidity of 35% or less, 30% or less, 25% or less, 20% or less, 15% or less, 10% or less, or 5% or less.

The manufacturing process may include a relatively high compression of granules to assemble a gum tablet of about 50 to 100 kN, 70 to 90 kN, or 60 to 80 kN.

Prior to compression, sweeteners, flavorants, fillers, etc., are added to the gum base/composition separately, i.e. in a dough mixer, Banbury mixer, or the like. In various embodiments, the gum/composition is pressed as contacting strips, a first strip containing the catalyst, and a second strip containing the peroxide. Prior to contacting the two strips to form an integral “stick” product, a barrier layer of a water-soluble polymer may be applied between the strips if necessary. In further embodiments, a third strip, containing no catalyst and no peroxide source is added between first and second strips. The strips thus pressed may be self-adherent or may require application of pressure, i.e. by passing through one or more roll nips, to produce a stick which will not easily separate into its component strips.

In various embodiments, a single strip is used, which may also take the form of a lozenge or other shape, for example by a pelletization process. In these embodiments, the separation of catalyst and peroxide may be accomplished by encapsulating one or the other of these components, or both components, prior to admixture to the gum base. In addition to encapsulation, one of the catalyst or peroxide may also be dispersed, generally uniformly, into a polymer which will liberate the component during mastication, in an aqueous environment. For example, a water-soluble polymer such as a polyacrylic acid or salt thereof can be used for this purpose.

Stated otherwise, the first and second portions are arranged to contact each other in such a manner that the peroxide source and the peroxide decomposition catalyst do not come into contact until the gum/oral/topical composition formulation is used. The first and second portions may thus have subportions. For example, if the first and second portions are arranged as layers, the peroxide source may be in a sublayer most distant from the second portion containing the peroxide decomposition catalyst. Alternatively, while the first layer may be thoroughly intermixed such that the peroxide source is well distributed throughout the first layer, the second layer may include at least two sublayers: upper and lower sublayers. The upper sublayer, which is not in contact with the first layer, may include the peroxide decomposition catalyst. The lower sublayer may contain all other components except for the catalyst. The lower sublayer may be placed between the first layer and the upper sublayer of the second layer. Alternatively still, the second layer may be a homogenous layer, having all components distributed throughout while the first layer may feature an upper sublayer containing the peroxide and a lower sublayer, in contact with the second layer, not including the peroxide source.

Alternatively still, the first and second layers may be divided by a third layer, for example composed of gumbase and being free of the peroxide source and the peroxide decomposition catalyst.

The individual layers may contact each other along a longitudinal axis of the layers or horizontal axis of the layers. Alternatively, the first portion and the second portion may alternate, with a third type of portion, free of the peroxide and the catalyst, may divide the first and second portions.

In yet another alternative embodiment, the catalyst and peroxide may be prepared and provided separately, for example as chewing gum pellets. For example, a first type of pellet may contain the catalyst and a second type of pellet may contain the peroxide. The first and second type of pellets may have the same or different color, shape, weight, texture, dimensions, the like, or a combination thereof. For ease of consumption, the two types of pellets may be differentiated, for example, by color or texture, such that a user can easily determine each type of the pellet to masticate. Determination may be made visually and/or by touch. The same or different amount of each type of pellets may be metered into a common packaging. The packaging may contain the pellets mixed or in separate compartments. The catalyst and the peroxide combine upon mastication when at least one pellet of each type is being masticated by the user at the same time.

When embodied in a candy or “soft candy” preparation, pellets, each containing but one of the catalyst and peroxide can be positioned adjacent each other in the form of the large “pellets” or intermixed with each other in granules, and encased with a hard shell.

The above modifications are also useful especially in gels, wherein the gel may contain one component of catalyst or peroxide, and the remaining component is contained in encapsulated form or dispersed in polymer pellets. Suitable gels are well known, and include those preparable from natural sources such as gelatins, starches, vegetable gums such as gum tragacanth or gum agar, etc. Synthetic gels such as those based on chemically modified celluloses, etc., may also be useful. The catalyst or peroxide, if the latter is liquid, may also be supplied as a complex with cyclodextrin. The chewable composition may conveniently be in cylindrical form as disclosed in U.S. Pat. No. 5,972,374.

When the composition is in the form of an oral lozenge, the same considerations apply as applied to the chewable compositions. The lozenges may be coated, i.e. with a soluble “protective” coating such as a polyvinyl alcohol, or with a candy coating. The lozenges contain at least one of the peroxide source and peroxide catalyst in an encapsulated form, or in another form which keeps the peroxide and catalyst separate until use. If encapsulated, the walls of the encapsulant are water soluble, so that they dissolve in the oral cavity, releasing their active ingredients. Wall materials of the encapsulated peroxide and/or catalyst may include coatings of sugars, starches, gelatins, or water soluble synthetic polymers such as polyvinyl alcohols, polyvinylpyrollidones, polyacrylic acids, and the like. Of course, if the lozenge is also masticated, mastication will enhance freeing of the encapsulated peroxide or catalyst. But since lozenges are ordinarily placed in the mouth and manipulated by the tongue, etc., their principle mode of use involves dissolution rather than mastication. As the encapsulated component(s) dissolve, the catalyst and peroxide come into contact, generating molecular oxygen. The lozenge may be in the form of a hard candy, for example by incorporating a relatively large amount of a sweetener such as xylitol, or may be in the form of a tablet composed of softer ingredients. In tablets, a larger amount of filler may advantageously be present. The filler may also serve as pH-adjusting material when appropriate. Calcium carbonate is such a filler, for example, which is mildly basic, while silicic acid, preferably in the form of precipitated or colloidal silica, is mildly acidic. A binder such as sugar, starch, gelatin, or adhesive polymer may be present.

The oral or topical composition may be included as a finished product packaged for sale, where the compositions are contained within or covered with a packaging of various embodiments. The packaging may include one or more materials capable of substantially preventing light from reaching the composition, material capable of substantially preventing the temperature of the composition from increasing or changing, or both. The material capable of substantially preventing or preventing light from reaching the composition may reflect or absorb light. Examples of such materials include reflective materials such as foils or opaque materials such as polyvinyl chloride. The material capable of substantially preventing light, compositional temperature change, or both may include materials that are insulative, having R-values that resist the flow of heat. Example insulative materials may include polyethylene, polyurethane, corrugated cardboard, or other materials known for storage of perishable items. The packaging should comply with food-grade requirements.

Besides a protective exterior as described above, the packaging may further include an interior for storing the composition and an openable and reclosable seal to the interior such as a zip, plastic zipper seal allowing a user access to the interior.

The present disclosure further relates to processes for preparing the composition or to the composition's use in mammalian species for elevating the oxygen content of the oral cavity. The composition may also be useful for veterinary purposes, i.e. for dogs, cats, horses, other hoofed animals, etc., as well as in humans. The composition may be used as a food additive or an animal health product for any type of animal with teeth including herbivores, carnivores, and omnivores. Overall ingredients and additives need to be chosen with respect to ingredient toxicity in animals such as avoiding ingredients which may harm a specific species. For example, the disclosed food additive meant for dog chow should be free of xylitol, which is toxic to dogs.

Further in at least one embodiment, a method of increasing molecular oxygen concentrations within an oral cavity is disclosed. The method may include chewing or masticating an oral composition of any embodiment described herein over a time period, wherein mastication causes the peroxide source and the vegetable- and/or fruit-based medium to come into contact with each other such that molecular oxygen is liberated by decomposition of the peroxide source and increases a concentration of molecular oxygen in an oral cavity over a time period. The time period differs and may depend on the intensity of mastication, the amount of the composition being masticated, and other factors. The oxygen generation may bring about an effervescent effect that can stimulate a sensory indication such as a tingling sensation, which can alert a user to the oxygen generation. Alternatively, a carbon dioxide producing additive such as carbonate crystal can provide a subjective perception of effervescence, simultaneous to the oxygen generation achieved upon mastication of the oral composition.

Alternatively, a method of increasing molecular oxygen concentration at a topical site is disclosed. The method may include topically applying a composition of any embodiment disclosed herein to a topical site. The method further includes applying moisture to the composition such that the moisture dissolves the composition and causes the peroxide source and the vegetable- or fruit-based medium to come into contact with each other such that molecular oxygen is liberated by decomposition of the peroxide source and substantially increases a concentration of molecular oxygen at the topical site for a time period. The time period may differ and is influenced by a variety of factors such as amount of applied composition, thickness and evenness of the film the composition forms at the site, the amount of moisture added, etc.

The topical place may be any place on or in the body. For example, the composition may be applied to skin or one or more mucous membranes. The composition may be epicutaneous. The composition may be applied to a surface of other tissue than skin. The topical composition may be in a form of a cream, foam, gel, lotion, ointment, liquid, powder, paste, tincture, or a combination thereof. Additionally, the composition may be incorporated in and applied as a transdermal patch.

The moisture added may be any fluid capable of increasing humidity at the application site. For example, while in the oral cavity, the saliva naturally moisturizes the composition, topical application may require application of a separate source of moisture. Example moisture source may be water. Water may be sterile, non-sterile, distilled, tap water, carbonated, mineral water, demineralized water. Various types of water or other fluid may be used, depending on the type of remedy needed at the application site.

In at least one embodiment, even the method of masticating the composition may include adding a separate moisturizer such as water. This may be especially the case when there is inadequate moisture present in the oral cavity.

In one or more embodiments, solid moisturizer may be added. For example, ice in the form of a sheet or cubes may be applied at the site to provide moisture as well as relief for swelling or another type of injury. Alternatively, a moisturizing gel, paste, or lotion may be applied over the composition.

The oral cavity or topical site may be anaerobic or have at least a partially anaerobic environment, where anaerobic is understood to mean an environment having molecular oxygen concentration suitable for colonization by anaerobic microorganisms or hypoxic/ischemic such that wound healing can be negatively affected.

One advantage of the methods of various embodiments is being able to provide oxygenation without perfusion (blood flow). This is a novel physiologic state that only exists in outer cell layers oxygenated by atmospheric oxygen via diffusion. The high concentration of dissolved molecular oxygen generated in the oral cavity or at the topical site delivers a much higher concentration of molecular oxygen and diffuses deeper into tissue than the amount of oxygen normally provided by gaseous oxygen (that may be inspired or provided by contact with the atmosphere). Perfusion relates to the passage of oxygen through the circulatory system to the tissue. Oxygenation without perfusion thus allows for the blood vessels regulating flow to vasoconstrict at the precapillary sphincter since oxygen supply is not needed there. This regulation is pH dependent and blood vessels' precapillary sphincters open when pH drops as cellular anaerobic metabolism creates lactic acid. Oxygenated cells do not need to rely on anaerobic glycolysis and don't make lactic acid so pH stays neutral as long as oxygen is there.

Further, the rapid production of oxygen for quick release may produce bubbling or a perception of a tingling effervescence. Slow production for sustained release may have no tactile perception, but rather a prolonged effect on the perioral tissues. The composition may have an initial effervescence, then a later sustained release by keeping some substrate or catalyst more concealed by a method such as microencapsulation, which may require more mastication to release the contents. The packaging may thus be designed with a specific effect of releasing a certain amount of oxygen at a rate. The rate may be constant or fluctuate such as increase or decrease during the time period.

In various embodiments, the increasing concentration of molecular oxygen in an oral cavity includes increasing the molecular oxygen concentration at surfaces of or within the oral mucosa, gums, cheeks, hard palate, soft palate, gingival crevices, teeth, tongue, or the deep crypts between the papillae. The increasing concentration of molecular oxygen in an oral cavity may include increasing the molecular oxygen concentration of gingival crevicular fluid, saliva, or any fluids including endogenously produced fluids or exogenous fluids within the oral cavity.

The increase of molecular oxygen in an oral cavity or at a topical site by, at least by, or the total concentration of molecular oxygen of an oral cavity or at a topical site during the increase may be about 1 parts per million (ppm), 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, 15 ppm, 16 ppm, 17 ppm, 18 ppm, 19 ppm, 20 ppm, 21 ppm, 22 ppm, 23 ppm, 24 ppm, 25 ppm, 26 ppm, 27 ppm, 28 ppm, 29 ppm, 30 ppm, 31 ppm, 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, 50 ppm, 51 ppm, 52 ppm, 53 ppm, 54 ppm, 55 ppm, 56 ppm, 57 ppm, 58 ppm, 59 ppm, 60 ppm, 61 ppm, 62 ppm, 63 ppm, 64 ppm, 65 ppm, 66 ppm, 67 ppm, 68 ppm, 69 ppm, 70 ppm, 71 ppm, 72 ppm, 73 ppm, 74 ppm, 75 ppm, 76 ppm, 77 ppm, 78 ppm, 79 ppm, 80 ppm, 81 ppm, 82 ppm, 83 ppm, 84 ppm, 85 ppm, 86 ppm, 87 ppm, 88 ppm, 89 ppm, 90 ppm, 91 ppm, 92 ppm, 93 ppm, 94 ppm, 95 ppm, 96 ppm, 97 ppm, 98 ppm, 99 ppm, 100 ppm, or more. In various embodiments, the increase in molecular oxygen concentration in an oral cavity or at a topical site by or the total concentration of molecular oxygen of an oral cavity or at a topical site may be a range between any two molecular oxygen concentrations listed above.

In various embodiments, a partial pressure of molecular oxygen (Po₂) or dissolved molecular oxygen concentration of an oral cavity or at a topical site during the increase in concentration of molecular oxygen may be about 33 mm Hg, 34 mm Hg, 35 mm Hg, 36 mm Hg, 37 mm Hg, 38 mm Hg, 39 mm Hg, 40 mm Hg, 41 mm Hg, 42 mm Hg, 43 mm Hg, 44 mm Hg, 45 mm Hg, 46 mm Hg, 47 mm Hg, 48 mm Hg, 49 mm Hg, 50 mm Hg, 51 mm Hg, 52 mm Hg, 53 mm Hg, 54 mm Hg, 55 mm Hg, 56 mm Hg, 57 mm Hg, 58 mm Hg, 59 mm Hg, 60 mm Hg, 61 mm Hg, 62 mm Hg, 63 mm Hg, 64 mm Hg, 65 mm Hg, 66 mm Hg, 67 mm Hg, 68 mm Hg, 69 mm Hg, 70 mm Hg, 71 mm Hg, 72 mm Hg, 73 mm Hg, 74 mm Hg, 75 mm Hg, 76 mm Hg, 77 mm Hg, 78 mm Hg, 79 mm Hg, 80 mm Hg, 81 mm Hg, 82 mm Hg, 83 mm Hg, 84 mm Hg, 85 mm Hg, 86 mm Hg, 87 mm Hg, 88 mm Hg, 89 mm Hg, 90 mm Hg, 91 mm Hg, 92 mm Hg, 93 mm Hg, 94 mm Hg, 95 mm Hg, 96 mm Hg, 97 mm Hg, 98 mm Hg, 99 mm Hg, 100 mm Hg, 101 mm Hg, 102 mm Hg, 103 mm Hg, 104 mm Hg, 105 mm Hg, 106 mm Hg, 107 mm Hg, 108 mm Hg, 109 mm Hg, 110 mm Hg, 111 mm Hg, 112 mm Hg, 113 mm Hg, 114 mm Hg, 115 mm Hg, 116 mm Hg, 117 mm Hg, 118 mm Hg, 119 mm Hg, 120 mm Hg, 121 mm Hg, 122 mm Hg, 123 mm Hg, 124 mm Hg, 125 mm Hg, 126 mm Hg, 127 mm Hg, 128 mm Hg, 129 mm Hg, 130 mm Hg, 131 mm Hg, 132 mm Hg, 133 mm Hg, 134 mm Hg, 135 mm Hg, 136 mm Hg, 137 mm Hg, 138 mm Hg, 139 mm Hg, 140 mm Hg, or more. In various embodiments, the Po₂ or dissolved molecular oxygen concentration of an oral cavity or at a topical site during the increase in concentration of molecular oxygen is a range between any Po₂ or dissolved molecular oxygen concentrations listed above.

The total amount of generated molecular oxygen is generated within the composition. After generation, the percentage of the total amount of generated molecular oxygen generated within the composition can diffuse from or escape the composition into the oral cavity or topical site. The percentage of the total amount of generated molecular oxygen generated within the composition may be at least or may be about 0.1%, 0.5%, 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% of the total amount of generated molecular oxygen. The percentage of the total amount of generated molecular oxygen generated within the composition is a range between any two percentages listed above.

The increase of molecular oxygen in the oral cavity or at the topical site, as compared to the state before mastication, application, or the peroxide source and the medium coming into contact at the application or mastication site, may be at least or may be about 0.1%, 0.5%, 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or any range of the numerals listed above.

The composition has the advantage that the oxygen liberated may change the metabolism of S. mutans to lessen production of lactic acid, without giving rise to drug resistant mutations as with the use of antibiotics. The disclosed composition has a further advantage that peroxide is present to act as a biocide, prior to its decomposition into oxygen. Thus, the use of the peroxide source with the catalyst provides a synergistic effect of reducing oral flora while also creating an aerobic environment.

As was mentioned above, the substrate and catalyst could be embodied in a lozenge or troche or a chewable/swallowable hard or soft candy. For veterinary applications, the components may be embodied within a rawhide or other long-lasting chew vehicle. Alternatively, the components may be provided as a food additive. For example, the peroxide source, the medium, or both components may be provided in a powder form which may be sprinkled, mixed in, or otherwise applied to/on the animal food. The size of the powder particles of the first component and the second component may differ or be the same. One or both of the components may be coated such that the active ingredients do not react with each other before an animal starts masticating the food the powder is applied onto. The components may be applied onto any type of animal food or mixed in the food. A non-limiting example of the food type may be dry chow, kibble, semi-moist food, or moist food.

Alternatively still, the powder may be pressed into granules or pellets, as was described above, which may be mixed into the animal feed, applied onto the animal food, or offered separately. The granules or pellets may be coated to be flavor-less or enhanced with one or more of the additional components named above, which are safe for the type of animal the pellets are being offered, and which may increase appeal. For example, the coating may be slightly sweet. The coating may be different for the first portion of the granules or pellets than for the second portion. The first portion may include the peroxide, the second portion may include medium. Additionally, the encapsulation and dispersion processes described above may be also utilized in the health product for veterinary use.

These oral embodiments rely on the agitation within the mouth to mix or combine the individual components. The oxygen produced is dissolved in water and as such will travel in the oral cavity fluid into interstices within the periodontal tissues. The substrate catalyst combination will become more in contact with progressively more chewing or manipulation within the mouth, until eventually all substrate will be converted to oxygenated water. Normal salivary pO₂ mirrors venous blood pO₂ and ranges from 33-44 mm Hg. 100% oxygen saturation in aqueous solution at body temperature occurs at about 120 mm Hg. Once that level is exceeded, oxygen bubbles will form and leave solution. Various embodiments can be designed that will release oxygen slowly or quickly, depending on the desired effect.

Regular use of these products may provide a pleasurable gum chewing experience while providing reduction in the incidence of tooth decay and anaerobe-based perioral diseases for the oral use. Alternatively, use of the composition for topical treatment may speed recovery of a wound or a lesion. Furthermore, the composition may aid in tooth decay, tooth loss, and gum disease prevention of various animal species.

In an alternative embodiment, a method of inhibiting lactic acid generation by microorganisms in an oral cavity is disclosed. The method includes masticating an oral composition of any embodiment disclosed herein over a time period, wherein mastication causes the peroxide source and the vegetable- or fruit-based medium to come into contact with each other such that molecular oxygen is liberated by decomposition of the peroxide source and increases a concentration of molecular oxygen in an oral cavity for the time period.

Likewise, a method for treating a wound, ailment, lesions, or another skin condition is disclosed. The method includes topically applying a composition of any embodiment disclosed herein to a wound or skin and applying moisture to the composition, wound, or skin, wherein the moisture dissolves the composition and causes the peroxide source and the vegetable- or fruit-based medium to come into contact with each other such that molecular oxygen is liberated by decomposition of the peroxide source and increases a concentration of molecular oxygen at the wound or skin site over a time period. The composition may have a bacteriostatic and bacteriocidal effect on any microorganisms present at the skin or wound. The wound may include a tissue normally covered by skin such as epidermis, dermis, subcutaneous tissues or hypodermis, or tissues beneath hypodermis.

The principles described above are illustrated by non-limiting examples of the composition listed below.

### EXAMPLES

### Example 1

Tables 1 and 2 disclose an ingredient listing of a non-limiting example of the disclosed composition, composed as a gum formulation having two portions arranged as layers. Table 1 lists ingredients, amounts, and weight percentages of individual components of the first portion or layer. Table 2 lists the same for the second portion or layer.

TABLE 1
First layer of the gum including the composition described herein.

| Component | Weight percent [wt. %] | Amount [mg] |
|---|---|---|
| Peroxide source(s) | 2.15 | 8.6 |
| Sweetener(s) | 95.8 | 383.2 |
| Flavoring(s) and colorant(s) | 2.05 | 9.8 |
| TOTAL | 100 | 401.6 |

TABLE 2
Second layer of the gum including
the composition described herein.

| Component | Weight percent [wt. %] | Amount [mg] |
|---|---|---|
| Peroxide decomposition | 0.23 | 2.07 |
| catalyst(s) | | |
| Gumbase(s) | 60 | 540.00 |
| Sweetener(s) | 32.97 | 296.73 |
| Flavoring(s) and colorant(s) | 4.3 | 38.7 |
| Anti-caking agent(s) | 2.00 | 18.00 |
| Nutritional supplement(s) | 0.5 | 4.5 |
| TOTAL | 100 | 900.0 |

TABLE 3
Non-limiting example components of the
first portion of the composition.

| Component | Weight percent [wt. %] | Amount [mg] |
|---|---|---|
| Calcium stearate | 1.5 | 6.00 |
| Carbamide | 0.65 | 2.60 |
| Sorbitol | 65.00 | 260.00 |
| Xylitol | 30.15 | 120.60 |
| Aspartame | 0.3 | 1.2 |
| Truvia | 0.2 | 0.8 |
| Sucralose | 0.1 | 0.4 |
| Acesulfame | 0.05 | 0.2 |
| Spearmint flavoring | 1.5 | 6.00 |
| Menthol flavoring | 0.25 | 1.00 |
| Vanilla flavoring | 0.3 | 2.6 |
| TOTAL | 100 | 401.6 |

TABLE 4
Non-limiting example components of the
second portion of the composition.

| Component | Weight percent [wt. %] | Amount [mg] |
|---|---|---|
| Red lentil powder | 0.23 | 2.07 |
| Gumbase | 60.00 | 540.00 |
| Sorbitol | 22.42 | 201.78 |
| Xylitol | 10.00 | 90.00 |
| Aspartame | 0.2 | 1.8 |
| Truvia | 0.1 | 0.9 |
| Sucralose | 0.1 | 0.9 |
| Acesulfame | 0.15 | 1.35 |
| Spearmint flavoring | 1.6 | 14.40 |
| Peppermint flavoring | 1.6 | 14.40 |
| Menthol flavoring | 0.4 | 3.60 |
| Vanilla flavoring | 0.7 | 6.3 |
| Calcium stearate | 2.00 | 18.00 |
| Zinc gluconate | 0.5 | 4.5 |
| TOTAL | 100 | 900.0 |

The ingredients of the first portion, shown in Tables 1 and 3, were mixed and homogenized together into a material which was shaped as a first layer. The ingredients of the second portion, shown in Tables 2 and 4, were mixed and homogenized together into a material which was shaped as a second layer. The first and second layers were then pressed together to form one object, a gum.

### Example 2

### Molecular Oxygen Production

The gum described in Example 1 was crushed in a paddle blender such as a STOMACHER for several minutes in 5 ml of an aqueous fluid at 25° C. without light or heat exposure. The aqueous fluid was either tap water (not having a catalase) or saliva (containing catalase and/or other natural peroxides). The paddle blender was used to mimic the process of the mastication. The concentration of molecular oxygen was measured with a CORNING model 317 Oxygen analyzer samples fluid. All data points represent duplicate measurements.

TABLE 5
A comparison of molecular oxygen generation before
mastication and in a time period of 5 minutes
after initiating simulated mastication.

| | Baseline O₂ Concentration | Peak O₂ Concentration |
|---|---|---|
| Fluid Type | [ppm] | [ppm] |
| Tap water | 5 | 14 |
| Saliva | 3 | 15 |

As shown in Table 5, the combination of actives lead to significant molecular oxygen production within 5 minutes.

TABLE 6
Molecular oxygen production over a time period for
the gum formulation of Example 1 in tap water.

| Mastication Duration [min] | O₂ Production [ppm] |
|---|---|
| Baseline - 0 | 5 |
| 2 | 13 |
| 4 | 14 |
| 10 | 12 |
| 15 | 8 |

As shown in Table 6, molecular oxygen production rose to a peak molecular oxygen concentration and declined after reaching the peak molecular oxygen concentration. The peak oxygen production was achieved at approximately 5-10 minutes.

### Example 3

### Shelf Life Analysis of the Gum Formulation of Example 1

A timed study was conducted to look at accelerated product shelf life and effect of heat (40° C.) and light exposure on the product of Example 1. The light exposure was assessed at 0, 2, 4, and 6 weeks of light exposure. All data points represent duplicate measurements. Table 7 shows the results from the timed study.

TABLE 7
Light and heat study of the formulation of Example
1 at 40° C. with and without light exposure.

| Length of | Light | Baseline Molecular | Peak Molecular |
|---|---|---|---|
| the study | Exposure | Oxygen Concentration | Oxygen Concentration |
| [weeks] | (+/−) | [ppm] | [ppm] |
| 2 | + | 5 | 7 |
| 2 | − | 5 | 14 |
| 4 | + | 5 | 6 |
| 4 | − | 5 | 11 |
| 6 | + | 4 | 6 |
| 6 | − | 4 | 8 |

As shown in Table 7, exposure to heat and light resulted in the deterioration of active components such that less molecular oxygen was generated. Brown spots were also observed on the gum formulation, which indicated decomposition of the peroxide component.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An oral or topical composition comprising:
a peroxide source; and
a vegetable- or fruit-based peroxide medium configured as a peroxide decomposition catalyst capable of catalyzing decomposition of the peroxide source upon contact with the peroxide source to produce molecular oxygen, the peroxide source and the vegetable- or fruit-based medium being arranged in the composition such that substantial contact of the peroxide source and the peroxide decomposition catalyst is prevented prior to use of the composition.

2. The composition of claim 1, wherein the vegetable- or fruit-based medium includes a plurality of vegetable- or fruit-based mediums, wherein each of the plurality of vegetable or fruit based mediums is capable of catalyzing the decomposition of the peroxide source when contacting the peroxide source to produce molecular oxygen.

3. The composition of claim 1, wherein the peroxide source is about 0.1 to about 3.0 wt. %, based on the total weight of the composition.

4. The composition of claim 1, wherein the vegetable- or fruit-based medium is about 0.1 to about 3.0 wt. %, based on the total weight of the composition.

5. The composition of claim 1, wherein the substantial contact is prevented in an aqueous environment.

6. The composition of claim 1, wherein the vegetable- or fruit-based medium includes a medium derived from red lentils and/or bananas.

7. The composition of claim 1, wherein the composition is a powder.

8. The composition of claim 1, wherein the composition is an animal food additive.

9. The composition of claim 1, wherein the composition is enclosed in a packaging comprising a material capable of substantially preventing light from reaching the composition and/or thermally insulative material.

10. A method of increasing molecular oxygen concentrations within an oral cavity, the method comprising:
masticating the composition of claim 1 over a time period, wherein mastication causes the peroxide source and the vegetable- or fruit-based medium to come into contact such that molecular oxygen is liberated by decomposition of the peroxide source and increases concentration c1 of molecular oxygen in an oral cavity over the time period to c2, c2>c1.

11. The method of claim 10, wherein c2 is greater than about 20 ppm of molecular oxygen over the time period.

12. A method of inhibiting lactic acid generation by anaerobic microorganisms in an oral cavity, the method comprising:
masticating the composition of claim 1 over a time period, wherein mastication causes the peroxide source and the vegetable- or fruit-based medium to come into contact such that molecular oxygen is liberated by decomposition of the peroxide source and increases concentration c1 of molecular oxygen in an oral cavity over the time period to c2, c2>c1.

13. A method of treating a wound or a skin condition, the method comprising:
topically applying the composition of claim 1 to a wound or skin and increasing moisture of the composition, wound, and/or skin such that the moisture dissolves the composition and causes the peroxide source and the vegetable- or fruit-based medium to come into contact such that molecular oxygen is liberated by decomposition of the peroxide source and increases concentration c1 of molecular oxygen at the wound or skin over the time period to c2, c2>c1.

14. A masticatable gum composition capable of releasing molecular oxygen into an oral cavity during mastication, the composition comprising:
a first component having a gum base and one of a peroxide source or a vegetable- or fruit-based medium, the vegetable- or fruit-based medium capable of catalyzing decomposition of the peroxide source when contacting the peroxide source to produce molecular oxygen; and
a second component in contact with the first component and having the other of the peroxide source or the vegetable- or fruit-based medium,
wherein the first and second components are arranged to prevent substantial contact of the peroxide and the vegetable- or fruit-based medium prior to mastication of the masticatable gum.

15. The composition of claim 14, wherein the second component further comprises a gum base.

16. The composition of claim 14, wherein the second component is a coating substantially covering an outer surface of the first component.

17. The composition of claim 14, wherein the second component has a gum base.

18. The composition of claim 14, wherein the composition is enclosed in a packaging comprising a material capable of substantially preventing light from reaching the composition.

19. The composition of claim 18, wherein the material includes at least one of a reflective or opaque material.

20. The composition of claim 18, wherein the composition is treated with gaseous nitrogen prior to placement in a packaging.
